(19) United States
(12) Patent Application Publication
(10) Pub. No.: US 2023/0190172 A1
(43) Pub. Date: Jun. 22, 2023

(54) METHOD AND APPARATUS WITH SIGNAL PROCESSING

(71) Applicant: Samsung Electronics Co., Ltd., Suwon-si (KR)

(72) Inventors: Junyeub SUH, Seoul (KR); Chisung BAE, Yongin-si (KR)

(73) Assignee: Samsung Electronics Co., Ltd., Suwon-si (KR)

(21) Appl. No.: 17/948,551

(22) Filed: Sep. 20, 2022

(30) Foreign Application Priority Data
Dec. 17, 2021 (KR) 10-2021-0182047

(51) Int. Cl. A61B 5/372

(52) U.S. Cl. CPC A61B 5/372

(57) ABSTRACT

A method and apparatus of processing a signal, where the method may include receiving spike signals of neurons detected by each of a plurality of electrodes, grouping electrodes, from among the plurality of electrodes, corresponding to spike signals generated from a neuron of the neurons into a group, calculating weights corresponding to the grouped electrodes, and processing signals for each of the neurons based on the weights.

### CROSS-REFERENCE TO RELATED APPLICATIONS

This application claims the benefit under 35 USC § 119(a) of Korean Patent Application No. 10-2021-0182047, filed on Dec. 17, 2021, in the Korean Intellectual Property Office, the entire disclosure of which is incorporated herein by reference for all purposes.

### BACKGROUND

### 1. Field

The following description relates to a method and apparatus of processing signals of neurons.

### 2. Description of Related Art

Electrodes may be used to measure a human biosignal. For example, signals of neurons may be measured to analyze human brainwaves, and electrodes may be used to measure the signals of the neurons. In order to measure signals of more neurons, the size of the electrodes may be reduced, or the electrodes may be used in the form of an array. However, when the size of the electrodes is reduced, various problems may occur, such as the signals of neurons not being accurately measured by the electrodes.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, here is provided a processor-implemented method of processing a signal, the method including receiving spike signals of neurons detected by each of a plurality of electrodes, grouping electrodes, from among the plurality of electrodes, corresponding to spike signals generated from a neuron of the neurons into a group, calculating weights corresponding to the grouped electrodes, and processing signals for each of the neurons based on the weights.

The grouping of the electrodes may include selecting spike signals having an amplitude exceeding a threshold from among the spike signals, determining target electrodes corresponding to the spike signals generated from the neuron using a covariance among the detected spike signals, and grouping the target electrodes into the group.

The determining of the target electrodes may include determining the target electrodes corresponding to spike signals having the covariance greater than a threshold.

The grouping of the electrodes may include selecting spike signals having an amplitude exceeding a threshold from among the spike signals, and determining target electrodes corresponding to spike signals having a maximum amplitude appear within a time period, and grouping the target electrodes into the group.

The calculating of the weights may include calculating the weights for each of the grouped electrodes based on amplitudes of the spike signals corresponding to the grouped electrodes.

The calculating of the weights may include calculating a sum of the amplitudes of the spike signals corresponding to the grouped electrodes, and calculating a weight corresponding to an electrode from among the grouped electrodes, based on a ratio between an amplitude of a spike signal corresponding to the electrode and the sum of the amplitudes.

The calculating of the weights may include calculating the weights based on whether the grouped electrodes correspond to an existing group.

The calculating of the weights may include, in response to the grouped electrodes not corresponding to the existing group storing information on electrodes grouped into a new group, and calculating weights for each of the electrodes grouped into the new group.

The calculating of the weights may include calculating the weights based on an average value of spike signals detected by the grouped electrodes, in response to the grouped electrodes corresponding to the existing group.

The processing of the signals for each of the neurons may include processing the signals for each of the neurons by calculating a weighted sum obtained by applying the weights to corresponding spike signals.

In another general aspect, here is provided an apparatus for processing a signal, the apparatus including a plurality of electrodes configured to process a signal of a user, a communication interface configured to receive spike signals of neurons detected by each of the plurality of electrodes, and a processor configured to group electrodes, from among the plurality of electrodes, corresponding to spike signals generated from a neuron of the neurons into a group, calculate weights corresponding to the grouped electrodes, and process signals for each of the neurons based on the weights.

The processor may be configured to select spike signals having an amplitude exceeding a threshold from among the spike signals, determine target electrodes corresponding to the spike signals generated from the neuron using a covariance among the detected spike signals, and group the target electrodes into the group.

The processor may be configured to select spike signals having an amplitude that exceeds a threshold among the spike signals, and determine target electrodes corresponding to spike signals having a maximum amplitude appear within a time period, and group the target electrodes into the group.

The processor may be configured to calculate the weights for each of the grouped electrodes based on amplitudes of the spike signals corresponding to the grouped electrodes.

The processor may be configured to calculate a sum of the amplitudes of the spike signals corresponding to the grouped electrodes, and calculate a weight corresponding to an electrode from among the grouped electrodes, based on a ratio between an amplitude of a spike signal corresponding to the electrode and the sum of the amplitudes.

The processor may be configured to calculate the weights based on whether the grouped electrodes correspond to an existing group.

The processor may be configured to, in response to the grouped electrodes not corresponding to the existing group store information on electrodes grouped into a new group, and calculate weights for each of the electrodes grouped into the new group.

The processor may be configured to calculate the weights based on an average value of spike signals detected by the grouped electrodes, in response to the grouped electrodes corresponding to the existing group.

The processor may be configured to process the signals for each of the neurons by calculating a weighted sum obtained by applying the weights to corresponding spike signals.

Other features and aspects will be apparent from the following detailed description, the drawings, and the claims.

### BRIEF DESCRIPTION OF THE DRAWINGS

FIG. 1 illustrates an example of a size relationship and an arrangement relationship between a neuron and electrodes that detect a signal of the neuron.

FIG. 2 illustrates an example of an apparatus for processing signals.

FIG. 3 is a diagram illustrating an example of processing signals.

FIG. 4 is a diagram illustrating an example of grouping electrodes.

FIGS. 5A and 5B illustrate examples of processing signals of neurons detected by electrodes.

FIG. 6 illustrates an example of calculating a covariance among spike signals detected by electrodes.

FIG. 7 is a diagram illustrating an example of processing signals.

FIG. 8 is a diagram illustrating an example of processing signals.

FIG. 9 is a diagram illustrating an example of an apparatus for processing signals.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order.

The features described herein may be embodied in different forms and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when a component is described as being “connected to,” or “coupled to” another component, it may be directly “connected to,” or “coupled to” the other component, or there may be one or more other components intervening therebetween. In contrast, when an element is described as being “directly connected to,” or “directly coupled to” another element, there can be no other elements intervening therebetween.

The terminology used herein is for describing various examples only and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

Although terms such as “first,” “second,” and “third”, A, B, C, (a), (b), (c), or the like may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in the examples described herein may also be referred to as a second member, component, region, layer, or section without departing from the teachings of the examples.

Unless otherwise defined, all terms used herein including technical or scientific terms have the same meaning as commonly understood by one of ordinary skill in the art to which examples belong. Terms, such as those defined in commonly used dictionaries, are to be interpreted as having a meaning that is consistent with their meaning in the context of the relevant art, and are not to be interpreted in an idealized or overly formal sense unless expressly so defined herein.

The use of the term “may” herein with respect to an example or embodiment (e.g., as to what an example or embodiment may include or implement) means that at least one example or embodiment exists where such a feature is included or implemented, while all examples are not limited thereto.

Hereinafter, examples will be described in detail with reference to the accompanying drawings. When describing the examples with reference to the accompanying drawings, like reference numerals refer to like elements and a repeated description related thereto will be omitted.

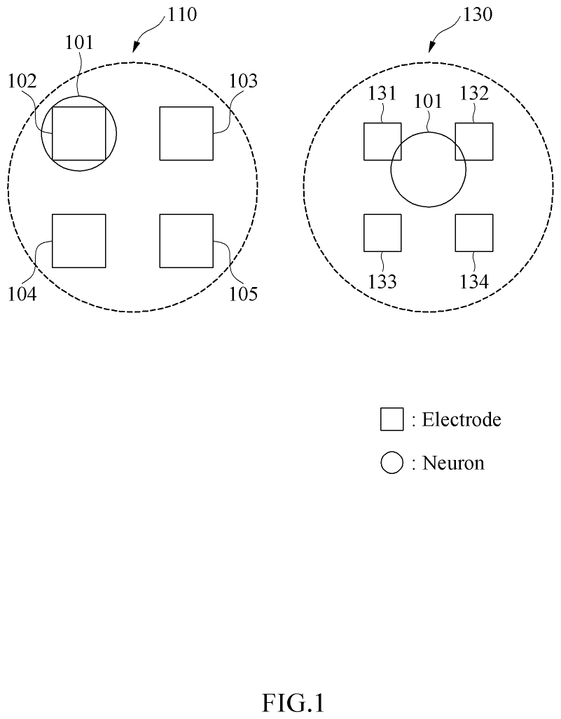

FIG. 1 illustrates an example of a size relationship and an arrangement relationship between a neuron and electrodes that detect a signal of the neuron. Referring to FIG. 1, reference numeral 110 denotes an arrangement of electrodes 102, 103, 104, and 105, of which electrode 102 cover a size of a neuron 101. Reference numeral 130 denotes an arrangement where sizes of electrodes 131, 132, 133, and 134 do not cover a size of the neuron 101.

In the drawing 110, when the sizes of the electrodes 102, 103, 104, and 105 sufficiently cover the size of the neuron 101, a single spike signal generated from the neuron 101 may be detected by a single electrode, that is, the electrode 102.

As shown in the drawing 130, when the sizes of the electrodes 131, 132, 133, and 134 are smaller than the sizes of the electrodes 102, 103, 104, and 105, the neuron 101 may not be completely covered by any one of the electrodes 131, 132, 133, and 134. In this case, the single spike signal generated from the neuron 101 may be detected by multiple electrodes, that is, the electrodes 131, 132, 133, and 134. However, for the spike signal of the neuron 101 measured by each of the electrodes 131, 132, 133 and 134, a signal-to-noise ratio (SNR) may vary according to a degree by which the electrodes 131, 132, 133, and 134 are displaced away from the neuron 101. In addition, when the neuron 101 is located away from the electrodes 131, 132, 133, and 134, the SNR of the spike signal of the neuron 101 decreases so that it may not be easy to detect the spike signal by an electrode at a location.

When the multiple electrodes, that is, the electrodes 131, 132, 133, and 134, detect the single spike signal generated from the neuron 101, the spike signals detected by the multiple electrodes, that is, the electrodes 131, 132, 133, and 134, may be utilized in an integrated manner to reduce noise generated from each of the electrodes 131, 132, 133, and 134, and to improve the SNR of the spike signal.

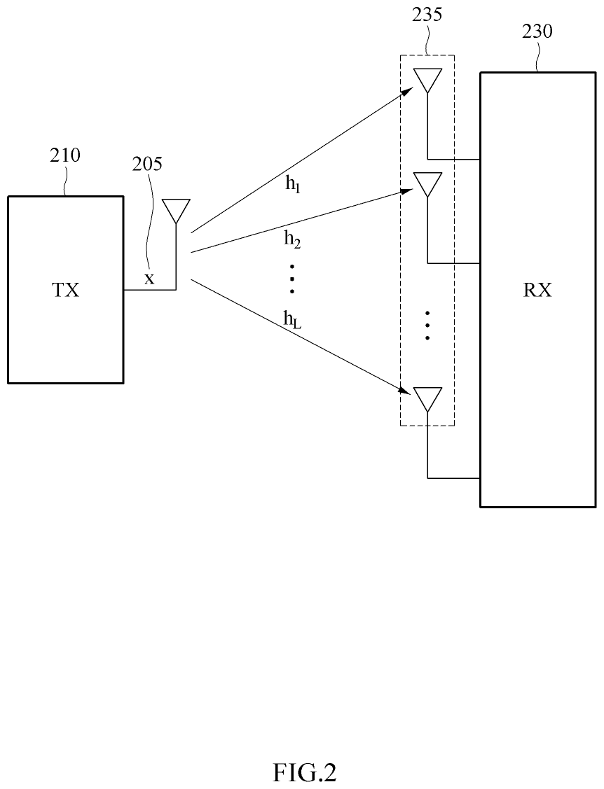

FIG. 2 illustrates an example of an apparatus for processing signals. Referring to FIG. 2, a transmission apparatus TX 210 and a reception apparatus RX 230 of a multiple input multiple output (MIMO) communication system are illustrated.

The transmission apparatus TX 210 may transmit a signal x 205, and the reception apparatus RX 230 may receive the signal x 205 through multiple antennas 235.

SNR of brainwaves may be improved by applying a maximal ratio combining (MRC) technique using multiple channels for improving an SNR of the MIMO communication system to a brainwave measurement system. In other words, the MRC technique may be applied to multiple electrode(s) in the brainwave measurement system to detect spike signal(s) generated from neuron(s).

The MRC technique may combine transformed transmission signals, which are received through the multiple antennas, using an optimal linear combination technique to improve a gain of a signal. Here, each channel characteristic (h1, h2, . . . , hL) may be added to the transmission signals. A gain of each channel may be proportional to an effective value (i.e., root mean square (RMS)) signal level and may be inversely proportional to a noise level that is a squared mean of a corresponding channel. In addition, different proportional constants may be used for each channel.

In an example, the signal x 205 transmitted by the transmission apparatus TX 210 is the aforementioned brainwave or spike signal generated from the neuron, and the multiple antennas 235 of the reception apparatus RX 230, which receive the signal x 205, are multiple electrodes receiving a single brainwave, and thus, it may be possible to improve the SNR of the brainwaves by applying the MRC technique.

In a diversity reception environment of the MIMO communication system, the reception apparatus RX 230 may receive multiple transformed transmission signals. In this case, a reception signal (Y) that a reception apparatus receives through the multiple antennas in the MIMO communication system may be represented by Equation 1 below.

y=hx+n [Equation 1]

Here, h may correspond to a matrix indicating a channel according to an antenna relationship of the MIMO communication system. In addition, X may denote the transmission signal transmitted by the transmission apparatus, and n may denote a reception noise.

If a weight according to the MRC technique is w^{H}, Equation 1 may be represented by Equation 2 below.

$$\begin{matrix}{w}^{H} y = {w}^{H} hx + {w}^{H} n \\ = {w}_{1} {h}_{1} x + {w}_{2} {h}_{2} x + … + {w}_{n} {h}_{n} x + {w}_{1} {n}_{1} + \\ {w}_{2} {n}_{2} + … + {w}_{n} {n}_{n}\end{matrix} \qquad [ Equation 2 ]$$

In addition, Equation 2 may be arranged with respect to a weight (W), as represented by Equation 3 below.

$$w = {argmax}_{w} \frac{{( {\sum }_{k = 1}^{L} {w}_{k} {h}_{k} x )}^{2}}{{( {\sum }_{k = 1}^{L} {w}_{k} {n}_{k} )}^{2}} \qquad [ Equation 3 ]$$

The signal processing apparatus may improve the SNR of the brainwaves by increasing a signal component (Σₖ₌₁^{L}wₖhₖx)² and reducing a noise component (Σₖ₌₁^{L}wₖhₖx)² in Equation 3. Here, the weight (W) may be calculated whenever the spike signal is input and grouped. The apparatus for processing signals may cumulatively store the weight (W).

Unlike the MIMO communication system in which a channel characteristic (H) of a complex channel is used, it may be assumed that the brainwave measurement system uses a scalar channel V, that is, a channel having different magnitudes of reception signals.

Accordingly, in the brainwave measurement system, even when a same spike signal is received, a magnitude of the spike signal may vary for each electrode. For example, when the same spike signal is received, the magnitude of the received spike signal may vary depending on a location of a corresponding electrode.

In other words, it may be understood that pieces of noise may be always the same but an SNR of each spike signal may vary in the brainwave measurement system. Therefore, the aforementioned Equation 1 may be modified in the brainwave measurement system as shown in Equation 4 below.

$$y = Vx + n \qquad [ Equation 4 ]$$

$$\to [ \begin{matrix}{y}_{1} \\ {y}_{2} \\ ⋮ \\ {y}_{L}\end{matrix} ] = [ \begin{matrix}{V}_{1} \\ {V}_{2} \\ ⋮ \\ {V}_{L}\end{matrix} ] x + [ \begin{matrix}{n}_{1} \\ {n}_{2} \\ ⋮ \\ {n}_{L}\end{matrix} ]$$

Here, y₁, y₂, . . . , y_{L} may correspond to signals received by respective L electrodes, and V₁, V₂, . . . , V_{L} may correspond to amplitude values of peaks of the signals received by the L electrodes. n₁, n₂, n_{L} may correspond to pieces of noise respectively corresponding to the L electrodes.

Equation 4 may be normalized as represented by Equation 5 below, for example.

$$[ \begin{matrix}\overline{{y}_{1}} \\ \overline{{y}_{2}} \\ ⋮ \\ \overline{{y}_{L}}\end{matrix} ] = \frac{1}{\sqrt{{\sum }_{k}^{L} {V}_{k}^{2}}} \{ [ \begin{matrix}{V}_{1} \\ {V}_{2} \\ ⋮ \\ {V}_{L}\end{matrix} ] x + [ \begin{matrix}{n}_{1} \\ {n}_{2} \\ ⋮ \\ {n}_{L}\end{matrix} ] \} \qquad [ Equation 5 ]$$

When the Cauchy-Schwarz inequality is applied to the MRC technique, w=h may be established, accordingly,

$$\frac{1}{\sqrt{{\sum }_{k}^{L} {V}_{k}^{2}}}$$

corresponding to h in Equation 5 may be represented as an average value (w̅ᵢ̅) of the weight as shown in Equation 6 below.

$$\overline{{w}_{i}} = \frac{{V}_{i}}{\sqrt{{\sum }_{k}^{L} {V}_{k}^{2}}} \qquad [ Equation 6 ]$$

In this case, since the gain is different for each channel, the average value (w̅ᵢ̅) of the weight may include an amplitude value of a peak (Vᵢ) corresponding to a size of each channel.

When applying the MRC technique, a brainwave signal (y_{MRC}) received by the multiple electrodes of the brainwave measurement system may be represented by Equation 7 below.

$${y}_{MRC} = \overline{w} \overline{y} = \frac{{V}^{T}}{{\sum }_{k}^{L} {V}_{k}^{2}} ( Vx + n ) \qquad [ Equation 7 ]$$

Here, (Vx+n) may correspond to a signal and noise received by a corresponding electrode, and

$$\frac{{V}^{T}}{{\sum }_{k}^{L} {V}_{k}^{2}}$$

may correspond to a weight according to the MRC technique.

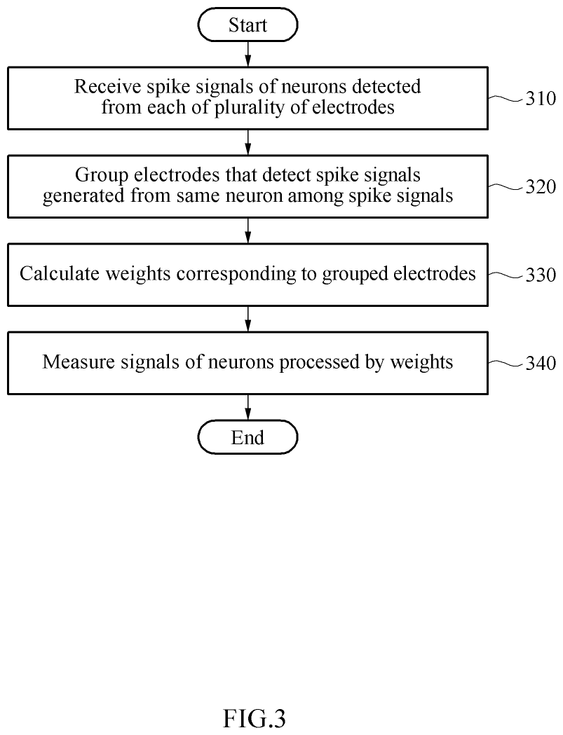

FIG. 3 is a diagram illustrating an example of processing signals. The operations in FIG. 3 may be performed in the sequence and manner as shown, although the order of some operations may be changed or some of the operations omitted without departing from the spirit and scope of the illustrative examples described. Many of the operations shown in FIG. 3 may be performed in parallel or concurrently. One or more blocks of FIG. 3, and combinations of the blocks, can be implemented by special purpose hardware-based computer, such as a processor, that perform the specified functions, or combinations of special purpose hardware and computer instructions. For example, an apparatus for processing a signal (hereinafter, “a signal processing apparatus”) may process signals for each of neurons through operations 310 to 340. In addition to the description of FIG. 3 below, the descriptions of FIGS. 1-2 are also applicable to FIG. 3, and are incorporated herein by reference. Thus, the above description may not be repeated here.

In operation 310, the signal processing apparatus may receive spike signals of neurons detected by each of a plurality of electrodes.

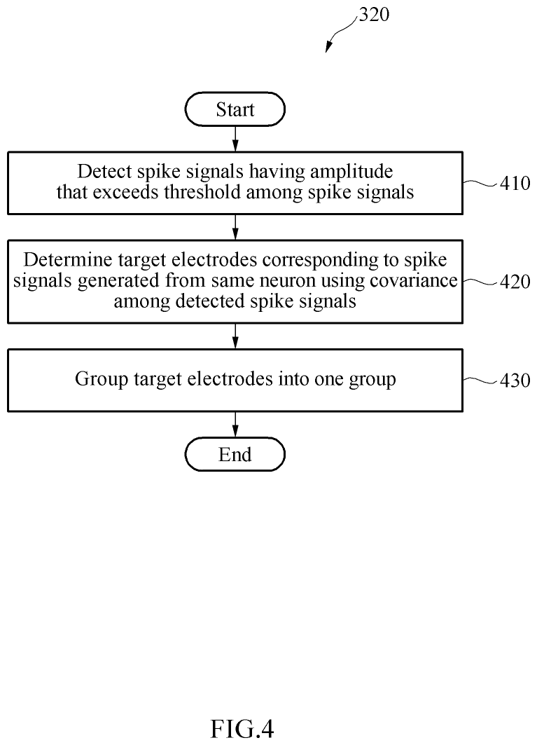

In operation 320, the signal processing apparatus may group electrodes respectively corresponding to spike signals generated from a same neuron among the spike signals received in operation 310 into one group. The signal processing apparatus may detect, for example, spike signals having an amplitude that exceeds a threshold among the spike signals. The signal processing apparatus may group target electrodes respectively corresponding to spike signals in which a time when a maximum amplitude appears at each of the detected spike signals is included in a same time period, into one group. Here, the target electrodes may be, for example, neighboring electrodes arranged adjacent to each other, in which spike signals having an amplitude that exceeds the threshold are detected in the same time period. An example in which the signal processing apparatus groups the electrodes into one group will be further described in more detail with reference to FIGS. 4 to 6 below.

In operation 330, the signal processing apparatus may calculate weights respectively corresponding to the electrodes grouped in operation 320. The signal processing apparatus may calculate the weights for each of the grouped electrodes based on amplitudes of the spike signals respectively corresponding to the electrodes grouped in operation 320. The signal processing apparatus may determine the weights for each of the electrodes to respectively correspond to magnitudes of the amplitudes of the spike signals detected by the grouped electrodes, for example. The signal processing apparatus may calculate a sum of the amplitudes of the spike signals respectively corresponding to the grouped electrodes. The signal processing apparatus may calculate a weight corresponding to one electrode among the grouped electrodes, based on a ratio between an amplitude of a spike signal corresponding to the one electrode and the sum of the amplitudes. In another example, the signal processing apparatus may calculate a weight corresponding to one electrode among the grouped electrodes, based on a ratio between an amplitude of a spike signal corresponding to the one electrode and the average of the amplitudes received at all the electrodes.

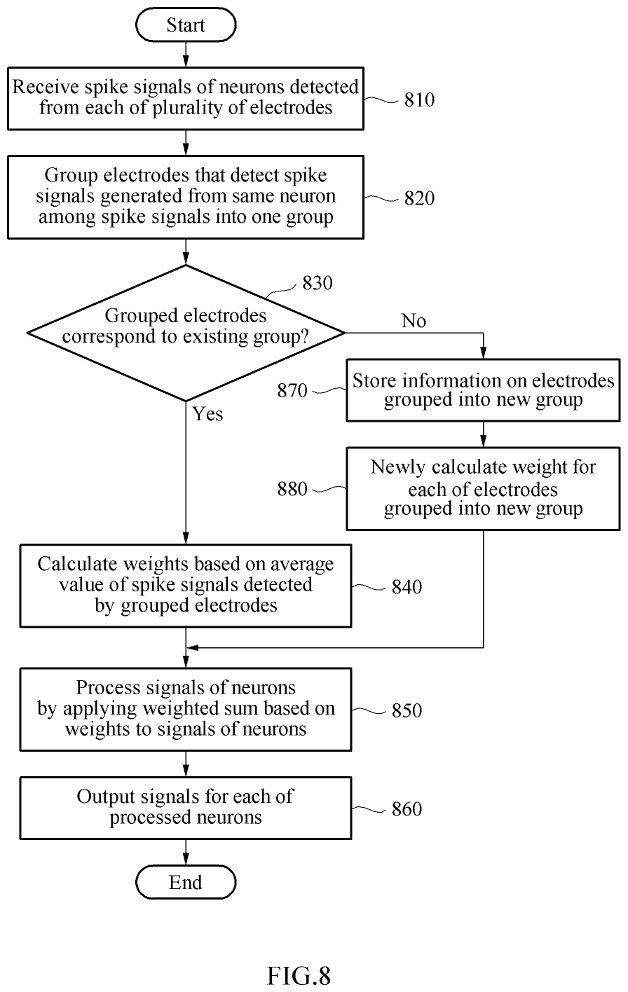

The signal processing apparatus may calculate the weights based on whether the grouped electrodes correspond to an existing group or a new group. An example in which the signal processing apparatus calculates the weights according to whether the grouped electrodes correspond to an existing group will be described in more detail with reference to FIG. 8 below.

In operation 340, the signal processing apparatus may process the signals for each of the neurons based on the weights calculated in operation 330. The signal processing apparatus may process, for example, the signals for each of the neurons by calculating a weighted sum obtained by applying the weights calculated in operation 330 to respective corresponding spike signals.

FIG. 4 is a diagram illustrating an example of grouping electrodes. The operations in FIG. 4 may be performed in the sequence and manner as shown, although the order of some operations may be changed or some of the operations omitted without departing from the spirit and scope of the illustrative examples described. Many of the operations shown in FIG. 4 may be performed in parallel or concurrently. One or more blocks of FIG. 4, and combinations of the blocks, can be implemented by special purpose hardware-based computer, such as a processor, that perform the specified functions, or combinations of special purpose hardware and computer instructions. For example, a signal processing apparatus may group electrodes into one group through operations 410 to 430. In addition to the description of FIG. 4 below, the descriptions of FIGS. 1-3 are also applicable to FIG. 4, and are incorporated herein by reference. Thus, the above description may not be repeated here.

In operation 410, the signal processing apparatus may detect spike signals having an amplitude that exceeds a threshold among the spike signals.

In operation 420, the signal processing apparatus may determine target electrodes respectively corresponding to spike signals generated from a same neuron by using a covariance among the spike signals detected in operation 410. For example, the signal processing apparatus may determine electrodes respectively corresponding to spike signals having the covariance among the spike signals detected in operation 410 greater than a threshold to be target electrodes to be grouped into one group, that is, the target electrodes that detect a same neuron signal.

In operation 430, the signal processing apparatus may group the target electrodes determined in operation 420 into one group.

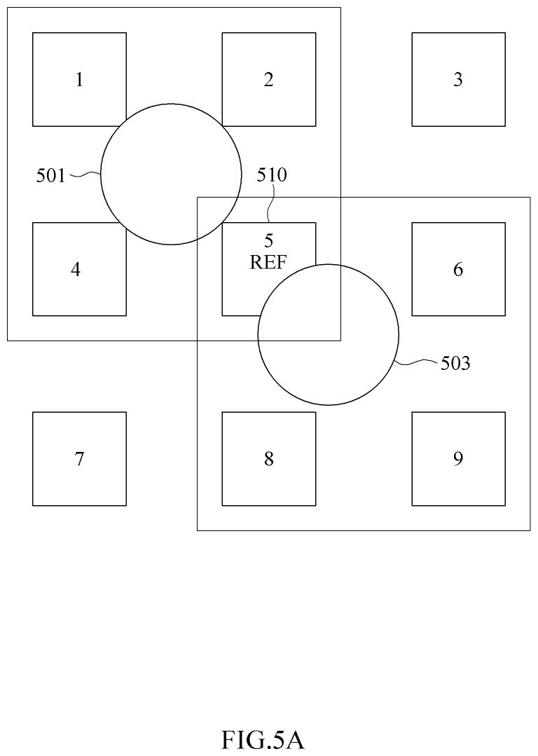

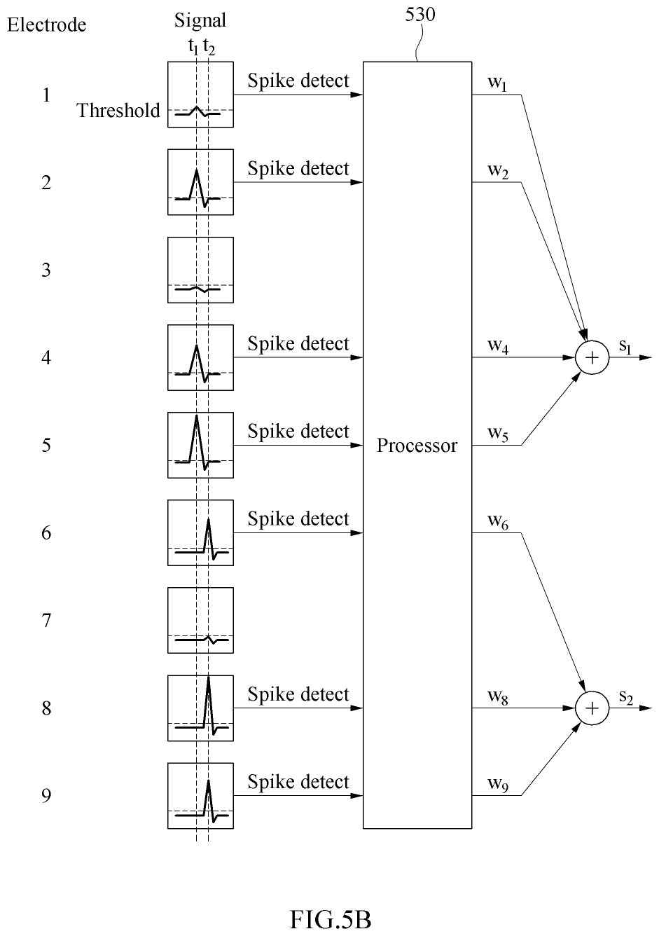

FIGS. 5A and 5B illustrate examples of processing signals of neurons detected by electrodes.

Referring to FIG. 5A, a situation in which multiple (e.g., nine) electrodes detect spike signals output from two neurons 501 and 503 is illustrated.

As described above, a spike signal generated from one neuron may be detected by multiple electrodes, but magnitudes of the spike signal may vary, or the spike signal may not be readily detected depending on locations of the respective electrodes. All electrodes that detect one common spike signal may be used to improve an SNR of the spike signal.

In an example, a signal processing apparatus may find electrodes that detect spike signals having a covariance greater than a threshold for each spike signal and group the electrodes into one group.

For example, when the spike signal is detected, the signal processing apparatus may determine whether the corresponding spike signal is detected by the electrodes previously grouped into one group based on data stored in a memory. The signal processing apparatus may determine, for example, whether the corresponding spike signal is detected by the previously grouped electrodes by searching for data most similar to the detected spike signal from among the data stored in the memory.

When the corresponding spike signal is not similar to the data stored in the memory, the signal processing apparatus may determine that the spike signal is generated by a neuron located at a new location. In this case, the signal processing apparatus may generate a new group by considering neighboring electrodes that detect the spike signals having the covariance greater than the threshold with respect to the corresponding spike signal to be one group that detects spike signals generated from a same neuron.

The signal processing apparatus may determine weights respectively corresponding to respective electrodes grouped into the new group in proportion to magnitudes (e.g., amplitudes) of the spike signals measured by the respective electrodes based on the detected spike signals. The signal processing apparatus may improve an SNR of a neuron signal by combining the spike signals based on the determined weights. The signal processing apparatus may store information on the new group. The information on the new group may include, for example, identification information of the electrodes grouped into the new group, amplitudes of the spike signals detected by the electrodes grouped into the new group, an average value of the amplitudes, and the weights corresponding to the electrodes grouped into the new group, but examples are not limited thereto.

As shown in FIG. 5A, electrodes selected as one group may vary according to the spike signals detected by the multiple electrodes. For example, when a spike signal is detected by a reference electrode REF 510, the signal processing apparatus may determine which of neurons 501 and 503 is the corresponding spike signal generated from based on a covariance among the corresponding spike signal and spike signals measured by the electrodes around the reference electrode REF 510. The signal processing apparatus may calculate a covariance among the spike signal sensed by a corresponding electrode and other spike signals sensed by neighboring electrodes to group electrodes from which a same spike signal is detected into one group.

The signal processing apparatus may obtain each weight based on an amplitude of a peak corresponding to power for grouped spike signals. The signal processing apparatus may obtain brainwaves with an improved SNR by calculating a weighted sum obtained by applying the weights to the corresponding spike signals.

Various brainwaves may be more accurately measured by improving SNRs of all measured brainwaves rather than classifying a particular type of brainwave.

Referring to FIG. 5B, an example of generating signals with an improved SNR by using multiple spike signals measured by nine electrodes is illustrated.

In an example, the signal processing apparatus may set a threshold in advance and, when a spike signal a greater than the threshold is detected by an arbitrary electrode, the reference electrode REF 510, may determine at a time when the spike signal is detected, if there are spike signals detected by the neighboring electrodes adjacent to the reference electrode REF 510 to be greater than the threshold.

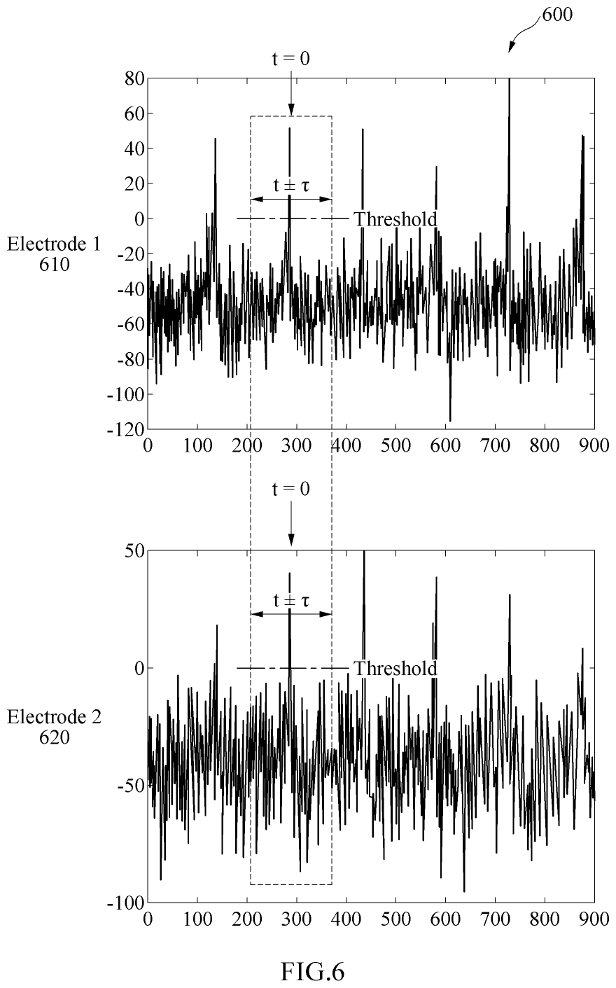

In an example, the signal processing apparatus may set a time interval (e.g., ±T) at a distance from a corresponding peak for each of the electrodes in a forward and backward direction, as shown in FIG. 6 below, and determine if there are spike signals detected by the neighboring electrodes in the time interval. The signal processing apparatus may obtain a covariance for each tau (T) based on the peak in the time interval (e.g., ±T) for each of the electrodes, and may group electrodes that may have a same stimulus into one group when the covariance is greater than a preset criterion. Here, the grouping may correspond to a process of mixing the spike signals.

The signal processing apparatus may improve the SNR of the brainwaves by applying MRC to the grouped spike signals. The process of calculating the covariance among the spike signals will be described in more detail with reference to FIG. 6 below.

In an example, when the spike signals are detected by the nine electrodes, a processor 530 of the signal processing apparatus may group electrodes corresponding to the spike signals. For example, a highest amplitude of spike signals detected by electrodes 3 and 7 among the nine electrodes may not reach the threshold. For example, the signal processing apparatus may detect spike signals of which peaks corresponding to highest amplitudes of the respective corresponding spike signals that exceed the threshold, such as electrodes 1, 2, 4, 5, 6, 8, and 9. Here, a time when the highest amplitudes appear at the spike signals detected by the electrodes 1, 2, 4, and 5 may be t1, and a time when the highest amplitudes appear at the spike signals detected by the electrodes 6, 8, and 9 may be t2.

The signal processing apparatus may group target electrodes corresponding to spike signals in which a time when a maximum amplitude appears at each of the detected spike signals is included in a same time period, into one group. The signal processing apparatus may group the electrodes 1, 2, 4, and 5 corresponding to the spike signals having the highest amplitudes in a time period based on the t1 into a first group. In addition, the signal processing apparatus may group the electrodes 6, 8, and 9 corresponding to the spike signals having the highest amplitudes in a time period based on the t2 into a second group.

For example, when the neuron 501 outputs spike signals at t1 and the neuron 503 outputs spike signals at t2, the first group may correspond to electrodes that detect neuron signals of the neuron 501 and the second group may correspond to electrodes that detect neuron signals of the neuron 503.

The signal processing apparatus may calculate a weight corresponding to each of the electrodes based on magnitudes of the spike signals in each of the grouped electrodes.

Referring to Equation 7 above, a weight (wᵢ) at an i-th electrode may be defined as a value obtained by dividing an amplitude Vᵢ of a spike signal detected by the i-th electrode by a squared sum (ΣⱼVₛₚᵢₖₑ²) of amplitudes of spike signals in a group to which the i-th electrode belongs as shown in Equation 8. Here, a may correspond to a constant value.

$${w}_{i} = α \frac{{V}_{i}}{{\sum }_{j} {V}_{spike}^{2}} \qquad [ Equation 8 ]$$

The signal processing apparatus may calculate the signals for each of the neurons (e.g., s1, s2) processed to reduce noise by a weighted sum obtained by multiplying the weight by a corresponding spike signal and adding up a result of the multiplying. A signal s1 may correspond to the neuron 501, and a signal s2 may correspond to the neuron 503.

FIG. 6 illustrates an example of calculating a covariance among spike signals detected by electrodes. Referring to FIG. 6, spike signals detected in a predetermined time interval (e.g., ±T) based on a point in time (t=0) are shown in an electrode 1 610 and an electrode 2 620. Here, the time point (t=0) of the electrode 1 610 appears to be similar to the time point (t=0) of the electrode 2 620, but a time difference may occur due to a capacitor component, and thus the electrode 1 610 and the electrode 2 620 may have different time axes.

A signal processing apparatus may obtain a covariance between the spike signal for each of the electrodes and a spike signal of another electrode having a peak detected around the corresponding electrodes in an interval (e.g., ±T) at a same distance from a time of a peak for the spike signal for each of the electrodes in a forward and backward direction. In an example, T may be obtained empirically.

The signal processing apparatus may calculate, for example, the covariance by Equation 9 below.

$$Cov ( d 1 , d 2 ) = \frac{{\sum }_{i = t - τ}^{t + τ} ( d {1}_{i} - \overline{d 1} ) ( d {2}_{i} - \overline{d 2} )}{2 τ} \qquad [ Equation 9 ]$$

Here, for example, d1 may correspond to an amplitude at a peak of a spike signal detected by the electrode 1 610, and d2 may correspond to an amplitude at a peak of a spike signal detected by the electrode 2 620.

In addition, d̅1̅ may correspond to an average value of spike signals detected by the electrode 1 610 during 2τ, and d̅2̅ may correspond to an average value of spike signals detected by the electrode 2 620 during 2τ.

When the covariance calculated by Equation 9 is greater than a threshold, the signal processing apparatus may determine that the spike signals detected by the electrode 1 610 and the spike signals detected by the electrode 2 620 are caused by a stimulus generated by a same neuron. Here, it may be understood that if the covariance between the two spike signals is greater than the threshold, tendency between the two spike signals may be same.

The signal processing apparatus may consider the electrodes that detect the spike signals having the covariance greater than the threshold to be the electrodes that receive a same stimulus, and thus group the electrodes into one group. The signal processing apparatus may improve the SNR of the brainwaves by applying MRC to the grouped spike signals.

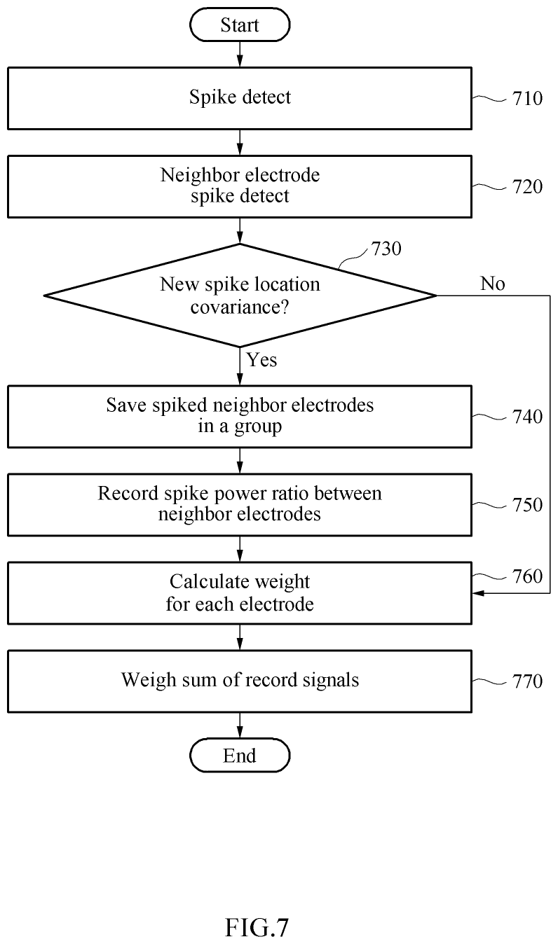

FIG. 7 is a diagram illustrating an example of processing signals. The operations in FIG. 7 may be performed in the sequence and manner as shown, although the order of some operations may be changed or some of the operations omitted without departing from the spirit and scope of the illustrative examples described. Many of the operations shown in FIG. 7 may be performed in parallel or concurrently. One or more blocks of FIG. 7, and combinations of the blocks, can be implemented by special purpose hardware-based computer, such as a processor, that perform the specified functions, or combinations of special purpose hardware and computer instructions. For example, a signal processing apparatus may obtain a brainwave signal with an improved SNR through operations 710 to 770. In addition to the description of FIG. 7 below, the descriptions of FIGS. 1-6 are also applicable to FIG. 7, and are incorporated herein by reference. Thus, the above description may not be repeated here.

In operation 710, the signal processing apparatus may detect a spike signal through a random electrode from among a plurality of electrodes.

In operation 720, the signal processing apparatus may detect spike signals from electrodes adjacent to the electrode from which the spike signal is detected in operation 710.

In operation 730, the signal processing apparatus may determine whether the spike signal detected in operation 710 and the spike signals detected in operation 720 correspond to spike signals of a same neuron or spike signals of a new neuron. The signal processing apparatus may determine whether the spike signals belong to the same neuron or different neurons by using a covariance among the spike signals.

In operation 730, when it is determined that the spike signals detected in operation 720 are spike signals of a new neuron corresponding to electrodes of a new group instead of an existing group, neighboring electrodes corresponding to the spike signals detected in operation 720 may be grouped into a new group in operation 740.

In operation 750, the signal processing apparatus may store information (e.g., a power ratio among the spike signals) on the spike signals corresponding to the electrodes grouped into the new group in operation 740.

In operation 760, the signal processing apparatus may calculate new weights respectively corresponding to each of the electrodes based on the power ratio among the spike signals stored in operation 750.

In operation 770, the signal processing apparatus may output summed signals by applying the new weights calculated in operation 760 to each of the spike signals.

In another example, in operation 730, when it is determined that the spike signals detected in operation 720 correspond to the electrodes in the existing group, in operation 760, the signal processing apparatus may calculate new weights corresponding to each of the electrodes by using the spike signals detected in operation 720 together with spike values that belong to the existing group.

In operation 770, the signal processing apparatus may output a weighted sum of signals by applying the new weights calculated in operation 760 to each of the spike signals.

FIG. 8 is a diagram illustrating an example of processing signals. The operations in FIG. 8 may be performed in the sequence and manner as shown, although the order of some operations may be changed or some of the operations omitted without departing from the spirit and scope of the illustrative examples described. Many of the operations shown in FIG. 8 may be performed in parallel or concurrently. One or more blocks of FIG. 8, and combinations of the blocks, can be implemented by special purpose hardware-based computer, such as a processor, that perform the specified functions, or combinations of special purpose hardware and computer instructions. For example, a signal processing apparatus may output a signal for each of neurons through operations 810 to 880. In addition to the description of FIG. 8 below, the descriptions of FIGS. 1-7 are also applicable to FIG. 8, and are incorporated herein by reference. Thus, the above description may not be repeated here.

In operation 810, the signal processing apparatus may receive spike signals of neurons detected by each of a plurality of electrodes.

In operation 820, the signal processing apparatus may group electrodes corresponding to spike signals generated from a same neuron among the spike signals received in operation 810 into one group.

In operation 830, the signal processing apparatus may determine whether the electrodes grouped in operation 820 correspond to an existing group.

When it is determined that the electrodes grouped in operation 820 correspond to the existing group, in operation 840, the signal processing apparatus may calculate weights based on an average value of the spike signals detected by the electrodes grouped in operation 820.

In operation 850, the signal processing apparatus may process the signal for each of the neurons by applying a weighted sum based on the weights calculated in operation 840 to the signal for each of the neurons.

In operation 860, the signal processing apparatus may output the signal for each of the neurons processed in operation 850.

In another example, when it is determined that the electrodes grouped in operation 820 do not correspond to the existing group, in other words, when it is determined that the electrodes correspond to a new group, in operation 870, the signal processing apparatus may store information on the electrodes grouped into the new group. In operation 880, the signal processing apparatus may newly calculate a weight for each of the electrodes grouped into the new group.

In operation 850, the signal processing apparatus may process the signal for each of the neurons by applying a weighted sum based on the weights newly calculated in operation 880 to the signal for each of the neurons.

In operation 860, the signal processing apparatus may output the signal for each of the neurons processed in operation 850.

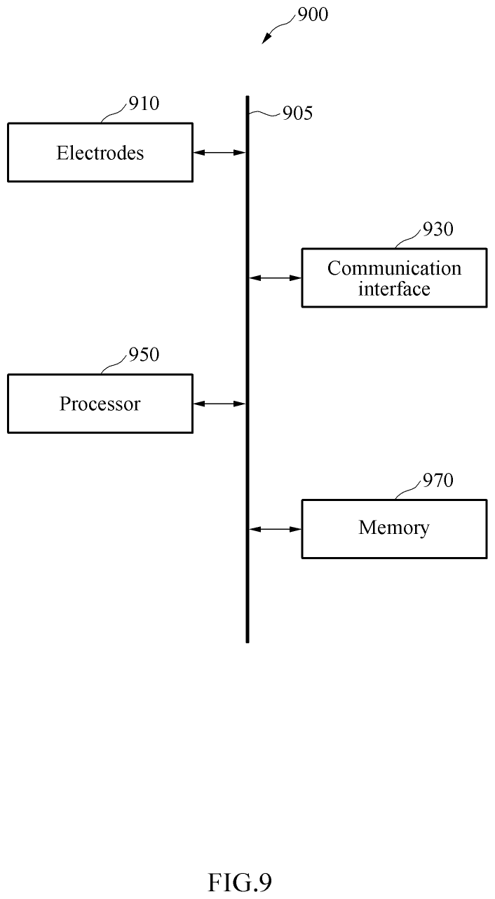

FIG. 9 is a diagram illustrating an example of a signal processing apparatus. Referring FIG. 9, a signal processing apparatus 900 may include electrodes 910, a communication interface 930, a processor 950, and a memory 970. The electrodes 910, the communication interface 930, the processor 950, and the memory 970 may be connected to each other through a communication bus 905.

The signal processing apparatus 900 may be, for example, a brainwave measurement system or a biosignal measurement apparatus, but examples are not necessarily limited thereto.

The electrodes 910 may detect a user's brainwaves.

The communication interface 930 may receive spike signals of neurons respectively detected by the electrodes 910. When the signal processing apparatus 900 does not include the electrodes 910, the communication interface 930 may receive the spike signals of the neurons from outside the signal processing apparatus 900.

The communication interface 930 may be construed to establish a direct (or wired) communication channel or a wireless communication channel between the signal processing apparatus 900 and an external electronic device and may support communication through the established communication channel. In an example, the communication device 660 may include a wireless communication module (e.g., a cellular communication module, a short-range wireless communication module, or a global navigation satellite system (GNSS) communication module) or a wired communication module (e.g., a local area network (LAN) communication module, or a power line communication (PLC) module). The wireless communication device may communicate with the external device via a short-range communication network (e.g., Bluetooth™, wireless-fidelity (Wi-Fi) direct, or IR data association (IrDA)) or a long-range communication network (e.g., a legacy cellular network, a 5G network, a next-generation communication network, the Internet, or a computer network (e.g., a LAN or a wide area network (WAN)).

The processor 950 may group electrodes corresponding to spike signals generated from a same neuron among the spike signals received through the communication interface 930. The processor 950 may calculate weights corresponding to the grouped electrodes. The processor 950 may process a signal for each of the neurons based on the weights.

In addition, the processor 950 may execute a program and control the signal processing apparatus 900. Program code to be executed by the processor 950 may be stored in the memory 970.

The memory 970 may store the spike signals received from the communication interface 930. The memory 970 may store information on the electrodes grouped into one group by the processor 950 and/or weights calculated by the processor 950 and corresponding to the grouped electrodes. Moreover, the memory 970 may store the signals for each of the neurons processed by the processor 950 based on the weights.

The memory 970 may store executable instructions to be executed by the processor 950. The memory 970 may store a variety of information generated from processing by the processor 950. In addition, the memory 970 may store a variety of data and programs. The memory 970 may include a volatile memory or a non-volatile memory. The volatile memory device may be implemented as a dynamic random-access memory (DRAM), a static random-access memory (SRAM), a thyristor RAM (T-RAM), a zero capacitor RAM (Z-RAM), or a twin transistor RAM (TTRAM).

The non-volatile memory device may be implemented as an electrically erasable programmable read-only memory (EEPROM), a flash memory, a magnetic RAM (MRAM), a spin-transfer torque (STT)-MRAM, a conductive bridging RAM (CBRAM), a ferroelectric RAM (FeRAM), a phase change RAM (PRAM), a resistive RAM (RRAM), a nanotube RRAM, a polymer RAM (PoRAM), a nano floating gate Memory (NFGM), a holographic memory, a molecular electronic memory device), or an insulator resistance change memory. Further details regarding the memory 620 is provided below.

In addition, the processor 950 may perform at least one of the methods described with reference to FIGS. 1 through 8 or a scheme corresponding to the at least one method. The processor 950 may be a hardware-implemented brainwave measurement system having a circuit that is physically structured to execute desired operations. For example, the operations may be implemented by execution of computer-readable instructions that configure the processing device to perform any one, or any combination, of the operations described above. The hardware-implemented signal processing apparatus, that is, the signal processing apparatus 900, may include, for example, a microprocessor, a central processing unit (CPU), a graphics processing unit (GPU), a processor core, a multi-core processor, a multiprocessor, an application-specific integrated circuit (ASIC), a field-programmable gate array (FPGA), and a neural processing unit (NPU).

The transmission apparatus TX 210, the reception apparatus RX 230, signal processing apparatus 900 and other apparatuses, devices, units, modules, and components described herein are implemented by hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, multiple-instruction multiple-data (MIMD) multiprocessing, a controller and an arithmetic logic unit (ALU), a DSP, a microcomputer, an application-specific integrated circuit (ASIC), a field programmable gate array (FPGA), a programmable logic unit (PLU), a central processing unit (CPU), a graphics processing unit (GPU), a neural processing unit (NPU), or any other device capable of responding to and executing instructions in a defined manner.

The methods that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

The Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In an example, the instructions or software includes at least one of an applet, a dynamic link library (DLL), middleware, firmware, a device driver, an application program storing the method of processing a signal. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access programmable read only memory (PROM), electrically erasable programmable read-only memory (EEPROM), random-access memory (RAM), magnetic RAM (MRAM), spin-transfer torque (STT)-MRAM, static random-access memory (SRAM), thyristor RAM (T-RAM), zero capacitor RAM (Z-RAM), twin transistor RAM (TTRAM), conductive bridging RAM (CBRAM), ferroelectric RAM (FeRAM), phase change RAM (PRAM), resistive RAM (RRAM), nanotube RRAM, polymer RAM (PoRAM), nano floating gate Memory (NFGM), holographic memory, molecular electronic memory device), insulator resistance change memory, dynamic random access memory (DRAM), static random access memory (SRAM), flash memory, non-volatile memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, blue-ray or optical disk storage, hard disk drive (HDD), solid state drive (SSD), flash memory, a card type memory such as multimedia card micro or a card (for example, secure digital (SD) or extreme digital (XD)), magnetic tapes, floppy disks, magneto-optical data storage devices, optical data storage devices, hard disks, solid-state disks, and any other device that is configured to store the instructions or software and any associated data, data files, and data structures in a non-transitory manner and providing the instructions or software and any associated data, data files, and data structures to a processor or computer so that the processor or computer can execute the instructions. In an example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A processor-implemented method of processing a signal, the method comprising:
receiving spike signals of neurons detected by each of a plurality of electrodes;
grouping electrodes, from among the plurality of electrodes, corresponding to spike signals generated from a neuron of the neurons into a group;
calculating weights corresponding to the grouped electrodes; and
processing signals for each of the neurons based on the weights.

2. The method of claim 1, wherein the grouping of the electrodes comprises:
selecting spike signals having an amplitude exceeding a threshold from among the spike signals;
determining target electrodes corresponding to the spike signals generated from the neuron using a covariance among the detected spike signals; and
grouping the target electrodes into the group.

3. The method of claim 2, wherein the determining of the target electrodes comprises determining the target electrodes corresponding to spike signals having the covariance greater than a threshold.

4. The method of claim 1, wherein the grouping of the electrodes comprises:
selecting spike signals having an amplitude exceeding a threshold from among the spike signals; and
determining target electrodes corresponding to spike signals having a maximum amplitude appear within a time period; and
grouping the target electrodes into the group.

5. The method of claim 1, wherein the calculating of the weights comprises calculating the weights for each of the grouped electrodes based on amplitudes of the spike signals corresponding to the grouped electrodes.

6. The method of claim 5, wherein the calculating of the weights comprises:
calculating a sum of the amplitudes of the spike signals corresponding to the grouped electrodes; and
calculating a weight corresponding to an electrode from among the grouped electrodes, based on a ratio between an amplitude of a spike signal corresponding to the electrode and the sum of the amplitudes.

7. The method of claim 1, wherein the calculating of the weights comprises:
calculating the weights based on whether the grouped electrodes correspond to an existing group.

8. The method of claim 7, wherein the calculating of the weights comprises, in response to the grouped electrodes not corresponding to the existing group:
storing information on electrodes grouped into a new group; and
calculating weights for each of the electrodes grouped into the new group.

9. The method of claim 7, wherein the calculating of the weights comprises calculating the weights based on an average value of spike signals detected by the grouped electrodes, in response to the grouped electrodes corresponding to the existing group.

10. The method of claim 1, wherein the processing of the signals for each of the neurons comprises processing the signals for each of the neurons by calculating a weighted sum obtained by applying the weights to corresponding spike signals.

11. A non-transitory computer-readable storage medium storing instructions that, when executed by a processor, cause the processor to perform the method of claim 1.

12. An apparatus for processing a signal, the apparatus comprising:
a plurality of electrodes configured to process a signal of a user;
a communication interface configured to receive spike signals of neurons detected by each of the plurality of electrodes; and
a processor configured to
group electrodes, from among the plurality of electrodes, corresponding to spike signals generated from a neuron of the neurons into a group,
calculate weights corresponding to the grouped electrodes, and
process signals for each of the neurons based on the weights.

13. The apparatus of claim 12, wherein the processor is further configured to:
select spike signals having an amplitude exceeding a threshold from among the spike signals;
determine target electrodes corresponding to the spike signals generated from the neuron using a covariance among the detected spike signals; and
group the target electrodes into the group.

14. The apparatus of claim 12, wherein the processor is further configured to:
select spike signals having an amplitude that exceeds a threshold among the spike signals; and
determine target electrodes corresponding to spike signals having a maximum amplitude appear within a time period, and
group the target electrodes into the group.

15. The apparatus of claim 12, wherein the processor is further configured to:
calculate the weights for each of the grouped electrodes based on amplitudes of the spike signals corresponding to the grouped electrodes.

16. The apparatus of claim 15, wherein the processor is further configured to:
calculate a sum of the amplitudes of the spike signals corresponding to the grouped electrodes; and
calculate a weight corresponding to an electrode from among the grouped electrodes, based on a ratio between an amplitude of a spike signal corresponding to the electrode and the sum of the amplitudes.

17. The apparatus of claim 12, wherein the processor is further configured to:
calculate the weights based on whether the grouped electrodes correspond to an existing group.

18. The apparatus of claim 17, wherein the processor is further configured to, in response to the grouped electrodes not corresponding to the existing group:
store information on electrodes grouped into a new group; and
calculate weights for each of the electrodes grouped into the new group.

19. The apparatus of claim 17, wherein the processor is further configured to:
calculate the weights based on an average value of spike signals detected by the grouped electrodes, in response to the grouped electrodes corresponding to the existing group.

20. The apparatus of claim 12, wherein the processor is further configured to:
process the signals for each of the neurons by calculating a weighted sum obtained by applying the weights to corresponding spike signals.
